(19) United States
(12) Patent Application Publication
(10) Pub. No.: US 2013/0052264 A1
(43) Pub. Date: Feb. 28, 2013

(54) CONTROLLED-RELEASE PHARMACEUTICAL COMPOSITION INCLUDING TAMSULOSIN OR PHARMACEUTICALLY ACCEPTABLE SALTS THEREOF, AND ORAL FORMULATION INCLUDING THE SAME

(71) Applicants: Ho-Jin Chung, Daejeon (KR); Sang-Yeob Park, Daejeon (KR); Chaul-Min Pai, Daejeon (KR)

(72) Inventors: Ho-Jin Chung, Daejeon (KR); Sang-Yeob Park, Daejeon (KR); Chaul-Min Pai, Daejeon (KR)

(73) Assignee: SAMYANG BIOPHARMACEUTICALS CORPORATION, Seoul (KR)

(21) Appl. No.: 13/695,814

(22) PCT Filed: Apr. 27, 2011
(86) PCT No.: PCT/KR2011/003098

§ 371 Date: Nov. 2, 2012

(30) Foreign Application Priority Data
May 4, 2010 (KR) 10-2010-0042055

(51) Int. Cl. A61K 9/58; A61K 9/32; A61P 13/02; A61K 9/08; A61K 31/18; A61K 9/14; A61K 9/16

(52) U.S. Cl. 424/452; 424/490; 424/497; 424/465; 514/603; 424/495; 424/494

(57) ABSTRACT

A controlled-release pharmaceutical composition including first and second groups of microparticles, each of the microparticles including a core including tamsulosin or pharmaceutically acceptable salts thereof, a controlled-release polymer coating layer formed on the core, and an enteric polymer outer layer formed on the controlled-release polymer coating layer, wherein the average thickness of the controlled-release polymer coating layer is different in each of the first and second groups of microparticles, and an oral formulation including the same, are provided. This pharmaceutical composition can easily control the extent of release of an active ingredient depending on changes in pH in the intestinal tract and the release pattern of the active ingredient in the small intestine, thus preventing the active ingredient from being rapidly transferred into the blood to thereby minimize side-effects, and maintaining the effective blood concentration of the active ingredient for a predetermined period of time. Furthermore, this composition can shield the bitter taste of the active ingredient even when exposed to the inside of the mouth, thus increasing the therapeutic effects for patients upon oral administration.

### TECHNICAL FIELD

The present invention relates to a controlled-release pharmaceutical composition including tamsulosin or pharmaceutically acceptable salts thereof as an active ingredient, and an oral formulation including the same.

### BACKGROUND OF ART

Dysuria that results from prostatomegaly is a disease that commonly occurs only in men. This disease takes place in such a manner that the outlet of the bladder is blocked due to an enlarged prostate, and the al receptor of the prostate is increased to thus induce an excess of prostatic smooth muscle contraction, and thereby upon excretion of urine the bladder muscle is thickened, and the inner pressure of the bladder is raised owing to strong contraction and thus the membrane between muscle fibers is subject to pressure. Upon urination, it shows the symptoms of urine not being efficiently discharged, frequent urination, the strength and thickness of the urine stream being reduced, the start of urination being delayed, etc.

Useful as an al blocker that is selective to the prostatic smooth muscle, tamsulosin is effective at treating dysuria due to benign prostate hyperplasia, and a typical oral dose thereof is 0.2˜0.8 mg/day. Tamsulosin is known to have high selectivity to the prostatic smooth muscle and low action on the blood to thereby remarkably decrease side-effects such as orthostatic hypotension and so on, unlike other al blockers such as doxazosin, terazosin, etc.

However, tamsulosin is administered in a small dose because of its very strong efficacy, and if the initial blood concentration of tamsulosin is excessively increased due to very fast absorption in vivo, side-effects such as orthostatic hypotension and so on may occur despite high selectivity to the prostatic smooth muscle. Thus, a preferred dosage form of tamsulosin is a delayed and sustained release type of controlled-release preparation.

However, in the case of a controlled-release tablet composed of a single system, the entire system may be broken because of the generation of specific causes or the lack of some components, and drug release takes place at one time undesirably incurring serious side-effects. In particular, patients who suffer from dysphagia are problematic because of discomfort in taking it.

In addition, some prior techniques related to tamsulosin disclose that the release of tamsulosin in the form of particles is controlled and it is placed in capsules and formulated into capsule preparations. However, in order to adjust the release of the drug into two stages, the case where the outermost coating layer of controlled-release particles is coated with a drug mixed with a polymer is limited in terms of its application range because, when this drug is prepared in the form of an orally disintegrating tablet instead of placing it in capsules, patients who taken such a drug perceive the bitter taste due to the release of the drug in the mouth.

Thus, research into a controlled-release composition, which may control side-effects due to rapid drug release, may stably control the release pattern of tamsulosin so as to sustain the effects of the drug, and may shield the bitter taste of tamsulosin for a predetermined period of time even when exposed to the inside of the mouth is still required.

### DETAILED DESCRIPTION OF THE INVENTION

### Technical Objectives

An embodiment of the present invention provides a controlled-release pharmaceutical composition which contains tamsulosin or pharmaceutically acceptable salts thereof as an active ingredient, may easily control the extent of release of the active ingredient depending on changes in pH in the intestinal tract and the release pattern of the active ingredient in the small intestine, and may maintain the effective blood concentration of the active ingredient for a predetermined period of time.

Another embodiment provides an oral formulation including the above pharmaceutical composition.

### Technical Solution

In order to accomplish the above objects, the present invention provides a pharmaceutical composition comprising a first group of microparticles and a second group of microparticles, each of the microparticles comprising a core including tamsulosin or pharmaceutically acceptable salts thereof, a controlled-release polymer coating layer formed on the core, and an enteric polymer outer layer formed on the controlled-release polymer coating layer, wherein an average thickness of the controlled-release polymer coating layer is different in each of the first group of microparticles and the second group of microparticles.

In the pharmaceutical composition, the first group of microparticles may have the average thickness of the controlled-release polymer coating layer of 1˜20 μm, and an average thickness ratio of the controlled-release polymer coating layer in the first group of microparticles and the second group of microparticles may be 1:1.2˜10.

The weight ratio of the first group of microparticles and the second group of microparticles may be adjusted so that a total weight ratio of tamsulosin or pharmaceutically acceptable salts thereof included in these two groups of microparticles falls in a range of 1:0.1˜10.

Also, the pharmaceutical composition may further comprise a third group of microparticles, each of the microparticles comprising a core including tamsulosin or pharmaceutically acceptable salts thereof, a controlled-release polymer coating layer formed on the core, and an enteric polymer outer layer formed on the controlled-release polymer coating layer, wherein an average thickness of the controlled-release polymer coating layer of the third group of microparticles is different from the average thickness of the controlled-release polymer coating layer of the first group of microparticles and the second group of microparticles.

In the pharmaceutical composition further comprising the third group of microparticles, the first group of microparticles may have an average thickness of the controlled-release polymer coating layer of 1˜20 μm, the average thickness ratio of the controlled-release polymer coating layer in the first group of microparticles and the second group of microparticles may be 1:1.2˜10, and the average thickness ratio of the controlled-release polymer coating layer in the second group of microparticles and the third group of microparticles may be 1:1.2˜5.

The weight ratio of the first group of microparticles, the second group of microparticles, and the third group of microparticles may be adjusted so that a total weight ratio of tamsulosin or pharmaceutically acceptable salts thereof included in these three groups of microparticles falls in a range of 1:0.1˜10:0.1˜10.

The average diameter of each of the groups of microparticles may be 10˜4500 μm.

The core of each of the groups of microparticles may have an average diameter of 5˜3000 μm.

The core may include one or more pharmaceutically acceptable inert seeds selected from the group consisting of an ion-exchange resin, silica, sugar spheres, and microcrystalline cellulose. In this case, the core may be formed by mixing tamsulosin or pharmaceutically acceptable salts thereof with the inert seed, by incorporating tamsulosin or pharmaceutically acceptable salts thereof in the inert seed, or by coating the surface of the inert seed with tamsulosin or pharmaceutically acceptable salts thereof.

The controlled-release polymer coating layer of the groups of microparticles preferably includes a water-insoluble polymer, and may further include one or more selected from the group consisting of a water-soluble polymer, an enteric polymer, and a gastric polymer, in addition to the water-insoluble polymer.

The controlled-release polymer coating layer of each of the groups of microparticles may have the same composition.

The enteric polymer outer layer of the groups of microparticles may include an enteric polymer, and may further include one or more selected from the group consisting of a water-insoluble polymer, a water-soluble polymer, and a gastric polymer, in addition to the enteric polymer.

Also, the pharmaceutical composition may have a dissolution pattern in which, according to a dissolution test method of Korean Pharmacopoeia 8^{th} revision (KP VIII) (a second paddle method: 100 rotations per min, 500 ml of dissolution solutions at pH 1.2 and pH 72), a) with respect to a dissolution solution at pH 1.2, the tamsulosin or pharmaceutically acceptable salts thereof are dissolved in an amount of 1˜30 wt % within 2 h, based on the total weight thereof; and b) with respect to a dissolution solution at pH 7.2, the tamsulosin or pharmaceutically acceptable salts thereof are dissolved in an amount of 10˜60 wt % within 30 min, and 30˜80 wt % within 1 h, and 50 wt % or more within 4 h, based on the total weight thereof.

In addition, the present invention provides an oral formulation comprising the above pharmaceutical composition.

The oral formulation may be a capsule, a tablet (a normal tablet, a double layer tablet, a chewable tablet, or an orally disintegrating tablet), a dry syrup formulation, a syrup, a jelly formulation, or a granule.

The oral formulation may include tamsulosin at 0.2˜0.8 mg per unit dosage form.

Hereinafter, a detailed description will be given of a controlled-release pharmaceutical composition, and an oral formulation including the same, according to the present invention.

Unless otherwise stated, some terms used in the entire specification may be defined as follows.

As used herein, the terms “include” or “contain” and any variations thereof are intended to include any element (or component) without particular limitation, and cannot be interpreted to exclude the addition of other elements (or components).

The term “active ingredient” may be tamsulosin (a base tamsulosin having no additional salts), pharmaceutically acceptable salts of tamsulosin, isomers of tamsulosin, or mixtures thereof.

The term “group of microparticles” is a generic name of a set, mixture, or population of microparticles in which a controlled-release polymer coating layer formed on a core has the same average thickness or the average thickness thereof falls within the equivalent range, and does not indicate only the case where microparticles agglomerate physically in a single group, but may exist in a state of being randomly mixed with microparticles of another group of microparticles.

The term “average thickness” of the polymer coating layer means that the thicknesses of the polymer coating layer of ten microparticles randomly selected from each group of microparticles are averaged. The average thickness may be measured by observing the fracture surface of a microparticle using a scanning electron microscope (SEM).

The term “controlled-release” means that the release of the contained active ingredient is controlled in a desired pattern, and includes controlled release, sustained release, delayed release, pulse release, or combinations thereof.

The term “enteric” means that the release of the contained active ingredient is limited in the gastrointestines at low pH, and preferably that only 30% or less of the contained active ingredient is released within 2 h in a pH 1.2 dissolution test.

The term “enteric controlled-release” means that the release of the contained active ingredient is limited in the gastrointestines at low pH and is controlled in a desired pattern in the intestinal tract (in particular, the small intestine) at approximately neutral pH, and includes controlled release, sustained release, delayed release, pulse release, or combinations thereof.

The term “gastric polymer” or “enteric polymer” indicates a pH-dependent polymer, and means a polymer (gastric) which dissolves under acidic conditions of pH of less than 5, or a polymer (enteric) which is stable under acidic conditions of pH of less than 5 and dissolves under weak acidic or neutral conditions of pH 5 or more.

### I. Controlled-Release Pharmaceutical Composition

Culminating in the present invention, intensive and thorough research into controlled-release pharmaceutical compositions including tamsulosin as an active ingredient, carried out by the present inventors, resulted in the finding that such a composition includes two or more groups of microparticles, each of the microparticles comprising a core including an active ingredient, a controlled-release polymer coating layer formed on the outer surface of the core, and an enteric polymer outer layer formed on the outer surface of the controlled-release polymer coating layer, wherein the average thickness of the controlled-release polymer coating layer is different in each of the two or more groups of microparticles, and the case where the average thickness of the controlled-release polymer coating layer of each of the groups of microparticles is adjusted may allow easy design of a composition wherein the control of the release pattern of the active ingredient is possible, and furthermore, the effective blood concentration of the active ingredient may be maintained for a predetermined period of time, and even when the composition is exposed to the inside of the mouth, it may shield the bitter taste of the active ingredient for a predetermined period of time, thus increasing therapeutic effects for patients upon oral administration.

According to an embodiment of the present invention, a pharmaceutical composition is provided, which comprises first and second groups of microparticles, each of the microparticles comprising a core including tamsulosin or pharmaceutically acceptable salts thereof, a controlled-release polymer coating layer formed on the core, and an enteric polymer outer layer formed on the controlled-release polymer coating layer, wherein the average thickness of the controlled-release polymer coating layer is different in each of the first and second groups of microparticles.

Depending on needs, the pharmaceutical composition of the invention may further comprise a third group of microparticles, each of the microparticles comprising a core including tamsulosin or pharmaceutically acceptable salts thereof, a controlled-release polymer coating layer formed on the core, and an enteric polymer outer layer formed on the controlled-release polymer coating layer, wherein the average thickness of the controlled-release polymer coating layer of the third group of microparticles is different from that of the coating layer of the first and second groups of microparticles.

Specifically, the pharmaceutical composition of the invention may include two or more groups of microparticles, and respective microparticles have the same configuration comprising a core including an active ingredient, and a controlled-release polymer coating layer and an enteric polymer outer layer which are sequentially formed on the outer surface of the core, and the average thickness of the controlled-release polymer coating layer is different in each of the groups of microparticles.

The core, the controlled-release polymer coating layer, and the enteric polymer outer layer in each of the microparticles are specified below.

Core Including Active Ingredient

The core is located at the innermost position of each of the microparticles included in the pharmaceutical composition of the invention, and includes, as the active ingredient, tamsulosin, pharmaceutically acceptable salts of tamsulosin, isomers of tamsulosin, or mixtures thereof.

The core may be composed exclusively of (1) the active ingredient, or may further include (2) a pharmaceutically acceptable inert seed.

In the case where the core may be composed exclusively of (1) the active ingredient, if the active ingredient is in powder form or has significantly various shapes, or the size variation of the powder is severe, such an ingredient cannot be adapted for the core. In this case, the active ingredient may be provided in the form of a sphere or a shape similar to the sphere via the GPCG-1 rotor system, wet or dry extrusion, spheronization, or granulation.

On the other hand, in the case where the core further includes (2) the pharmaceutically acceptable inert seed, the inert seed may not be particularly limited so long as it is typical in the art, but preferably includes one or more selected from the group consisting of an ion-exchange resin, silica, a sugar sphere, and microcrystalline cellulose.

Also, in the case where the core further includes the inert seed, the core may be provided in (2-1) a granular or particular form of a mixture comprising the active ingredient and the inert seed; (2-2) a form in which the active ingredient is incorporated in the inert seed; or (2-3) a form in which the active ingredient is applied on the surface of the inert seed, but the present invention is not limited thereto.

In the case where (2-2) the active ingredient is incorporated in the inert seed, the inert seed is preferably a porous material which has a spherical shape or a shape similar to a sphere, and more preferably includes an ion-exchange resin, silica (e.g. Zeosil®, Tixosil®, available from Rhodia Silica Korea Co. Ltd.), or mixtures thereof.

Also, in the case where (2-3) the active ingredient is applied on the surface of the inert seed, the inert seed is preferably a sugar sphere such as non-pareil sugar, or microcrystalline cellulose such as Celphere™ (Asahi Kasei), having a spherical shape or a shape similar to a sphere. In this case, coating may be carried out in such a manner that the active ingredient is dissolved in water or an organic solvent and then the resulting solution is sprayed onto the inert seed. As such, the core may be prepared using a coater, a fluidized bed coater, a fluidized bed processor, a fluidized bed granulator, etc., and more specifically a fluidized bed system with a bottom spray, a centrifugal granulator, Granurex® (available from Freund), etc. may be utilized.

On the other hand, although the core is not particularly limited in terms of shape, size, size variation and the like, it may be provided in the form of a sphere or a shape similar to the sphere so that the controlled-release polymer coating layer and the enteric coating layer, which will be described later, may be uniformly formed.

In an embodiment of the invention, the core has an average diameter of 5˜3000 μm, preferably 10˜1500 μm, and more preferably 15˜1000 μm. Particularly in the case of an orally disintegrating tablet or chewable tablet including the pharmaceutical composition of the invention, the average diameter of the core is preferably set to 30˜500 μm in order to minimize foreign body sensation in the mouth.

If the core is provided in the form of (2-3) the surface of the inert seed being coated with the active ingredient, the average diameter of the inert seed may be 2˜2900 μm, preferably 3˜1400 μm, and more preferably 4˜900 μm.

The average diameter of the core or the seed has to have a small variation so as to exhibit reproducible efficacy, and may be measured using a particle size analyzer, a microscope, or an image analyzer. Specifically, the diameter variation of the core is 1˜200 μm, preferably 1˜100 μm, and more preferably 1˜50 μm.

In the course of manufacturing the core using the above methods, a variety of biologically inert ingredients may be additionally used to achieve additional purposes, including coating efficiency, stability of active ingredient, outer appearance, color, protection, maintenance, coupling, improvements in performance and manufacturing processes, supplementary release control, etc.

The specific kinds of the biologically inert ingredients, usage methods, and the addition to the core of the invention may be easily implemented in light of the technical levels of those having ordinary knowledge in the art, and may be variously modified.

The biologically inert ingredient may be mixed together when the core is manufactured with the active ingredient, and may have little or insignificant influence on the release of the active ingredient. The case where the biologically inert ingredient has little or insignificant influence on the release of the active ingredient means that the extent of release of the drug changes in the range within ±10% over time because of inclusion of the biologically inert ingredient when the core including the active ingredient is subjected to a dissolution test in vitro, compared to the case where the biologically inert ingredient is not included. In this case, the core containing the active ingredient may exhibit the same or similar dissolution pattern compared to the case where the biologically inert ingredient is not included.

According to an embodiment of the invention, the biologically inert ingredient may include a saccharide, a sugar alcohol, a polymer, a colorant, a flavorant, a sweetener, a surfactant, a lubricant, a stabilizer, an antioxidant, a foaming agent, paraffin, wax, or a plasticizer.

The polymer that may be additionally included in the core may be a water-soluble polymer, a water-insoluble polymer, an enteric polymer, or a gastric polymer, and is more preferably a water-soluble polymer. The water-soluble polymer may be one or more selected from the group consisting of water-soluble cellulose ether, water-soluble polyvinyl derivatives, and alkylene oxide polymers. Specifically, the water-soluble polymer may be methylcellulose, hydroxypropyl cellulose, hydroxypropyl methylcellulose, polyvinylpyrrolidone, polyvinyl alcohol, polyethylene glycol, polypropylene glycol, etc., and is preferably hydroxypropyl methylcellulose or polyvinylpyrrolidone.

Also, the plasticizer that may be additionally included in the core may be used in an amount of 60 wt % or less based on the total dry weight of the polymer used in each of the microparticles.

Specifically, the plasticizer may be one or more selected from the group consisting of triethyl citrate, dibutyl phthalate, diethyl phthalate, dibutyl sebacate, diethyl sebacate, tributyl citrate, acetyl triethyl citrate, acetyl triethyl citrate, propylene glycol, triacetin, polyethylene glycol, cetyl alcohol, stearyl alcohol, and cetostearyl alcohol, and is preferably triethyl citrate, dibutyl phthalate, diethyl phthalate, dibutyl sebacate, diethyl sebacate, or mixtures thereof.

Also, the lubricant that may be additionally included in the core may be used in an amount of 0.001˜300 wt % based on the total dry weight of the polymer used in each of the microparticles. In order to manifest glidant or anti-tacking effects due to the addition of a lubricant, the amount of the lubricant may be set to 0.001 wt % or more based on the total dry weight of the polymer, and is preferably set to 300 wt % or less in consideration of the addition efficiency of the lubricant.

Specifically, the lubricant may be one or more selected from the group consisting of stearic acid, glyceryl behenate, glyceryl monostearate, magnesium stearate, calcium stearate, silicon dioxide, talc, and magnesium silicate.

Controlled-Release Polymer Coating Layer

The controlled-release polymer coating layer is formed on the outer surface of the core so as to control the release of the active ingredient.

In particular, the pharmaceutical composition of the invention is advantageous because it includes two or more groups of microparticles in which the average thicknesses of the controlled-release polymer coating layer are different, making it possible to more effectively control the release pattern of the active ingredient. Also the average thickness ratio of the controlled-release coating layer in the groups of microparticles and the weight ratio of the groups of microparticles (preferably the total weight ratio of the active ingredient included in the groups of microparticles) may be adjusted, whereby a pharmaceutical composition having an ideal release pattern may be more readily designed.

The microparticles having different thicknesses of the controlled-release polymer coating layer are described later, and the common configuration of the controlled-release polymer coating layer is mentioned below.

(Common Configuration of Controlled-Release Polymer Coating Layer)

According to an embodiment of the invention, the controlled-release polymer coating layer may be formed on the core via a coating process using a typical coater, a fluidized bed coater, a fluidized bed processor, or a fluidized bed granulator. Specifically, this process may be conducted by means of a fluidized bed system with a bottom spray, a centrifugal granulator, Granurex® (available from Freund), etc.

The controlled-release polymer coating layer preferably includes a water-insoluble polymer which is conventionally used in the art.

Specifically, the water-insoluble polymer may be water-insoluble cellulose acetate, a water-insoluble acrylic acid-based copolymer, or mixtures thereof, and is preferably one or more selected from the group consisting of ethylcellulose, an ethyl acrylate.methyl methacrylate.trimethylammonioethyl methacrylate chloride copolymer, a methyl methacrylate.ethyl acrylate copolymer, and polyvinylacetate.

Particularly, in an embodiment of the invention, the ethyl acrylate.methyl methacrylate.trimethylammonioethyl methacrylate chloride copolymer may be those having 8.85˜11.96% of ammonio methacrylate units (e.g. Eudragit® RL) or having 4.48˜6.77% of ammonio methacrylate units (e.g. Eudragit® RS), the methyl methacrylate.ethyl acrylate copolymer may be Eudragit® NE30D, Eudragit® NE40D, Eudragit® NM30D, or mixtures thereof, and the polyvinylacetate may be Kollicoat® SR30D. More preferably, the water-insoluble polymer may be an ethyl acrylate.methyl methacrylate.trimethylammonioethyl methacrylate chloride copolymer having 4.48˜6.77% of ammonio methacrylate units.

On the other hand, the controlled-release polymer coating layer may further include one or more selected from the group consisting of a water-soluble polymer, an enteric polymer, and a gastric polymer, in addition to the water-insoluble polymer, depending on the release pattern of the active ingredient included in the core or the form of the preparation.

The additional polymer components are not particularly limited so long as they are conventional in the art.

According to an embodiment of the invention, the water-soluble polymer may be one or more selected from the group consisting of water-soluble cellulose ether, a water-soluble polyvinyl derivative, and an alkylene oxide polymer, and is preferably one or more selected from the group consisting of methylcellulose, hydroxypropyl cellulose, hydroxypropyl methylcellulose, polyvinylpyrrolidone, polyvinyl alcohol, polyethylene glycol, and polypropylene glycol.

Also, the enteric polymer may be one or more selected from the group consisting of an enteric cellulose derivative, an enteric acrylic acid-based copolymer, an enteric maleic acid-based copolymer, and an enteric polyvinyl derivative, and is preferably one or more selected from the group consisting of hydroxypropyl methylcellulose acetate succinate, hydroxypropyl methylcellulose phthalate, hydroxymethyl ethylcellulose phthalate, cellulose acetate phthalate, cellulose acetate succinate, cellulose acetate maleate, cellulose benzoate phthalate, cellulose propionate phthalate, methylcellulose phthalate, carboxylmethyl ethylcellulose, ethylhydroxyethyl cellulose phthalate, a styrene.acrylic acid copolymer, a methyl acrylate.acrylic acid copolymer, a methyl acrylate.methacrylic acid copolymer, a butyl acrylate.styrene.acrylic acid copolymer, a methacrylic acid.methyl acrylate copolymer, a methacrylic acid.ethyl acrylate copolymer, a methyl acrylate.methacrylic acid.octyl acrylate copolymer, a vinyl acetate.maleic anhydride copolymer, an ethylene.maleic anhydride copolymer, a vinyl butyl ethermaleic anhydride copolymer, an acrylonitrile.methyl acrylate.maleic anhydride copolymer, a butyl acrylate.styrene.maleic anhydride copolymer, polyvinyl alcohol phthalate, polyvinylacetal phthalate, polyvinylbutyrate phthalate, and polyvinylacetoacetal phthalate.

Also, the gastric polymer may be one or more selected from the group consisting of a gastric polyvinyl derivative and a gastric acrylic acid-based copolymer, and is preferably one or more selected from the group consisting of polyvinylacetal diethylamino acetate, and a methyl methacrylate.butyl methacrylate.dimethylaminoethyl methacrylate copolymer.

On the other hand, in order to achieve additional purposes including coating efficiency, stability of active ingredient, outer appearance, color, protection, maintenance, coupling, improvements in performance and manufacturing process, or supplemental release control upon forming the controlled-release polymer coating layer, a variety of biologically inert ingredients may be additionally used.

The biologically inert ingredient may include a saccharide, a sugar alcohol, a polymer, a colorant, a flavorant, a sweetener, a surfactant, a lubricant, a stabilizer, an antioxidant, a foaming agent, paraffin, wax, or a plasticizer, and the detailed description thereof refers to the above description of the core.

(Groups of Microparticles having Different Average Thicknesses of Controlled-Release Polymer Coating Layer)

The pharmaceutical composition of the invention may include two or more groups of microparticles in which the average thicknesses of the controlled-release polymer coating layer are different. Accordingly, the release pattern of the active ingredient may be more effectively controlled, and the average thickness ratio of the controlled-release coating layer in the groups of microparticles and the weight ratio of the groups of microparticles (preferably the total weight ratio of the active ingredient included in the groups of microparticles) are adjusted, thereby more readily designing a pharmaceutical composition having an ideal release pattern.

If the average thickness of the controlled-release polymer coating layer is different in each of the groups of microparticles, release behavior of the active ingredient becomes different. Hence, a plurality of groups of microparticles in which the average thicknesses of the controlled-release polymer coating layer are different are manufactured, after which the release pattern of the active ingredient may be simulated via various combinations of the groups of microparticles (i.e. the average thickness ratio of the controlled-release coating layer in the groups of microparticles and the total weight ratio of the active ingredient included in the groups of microparticles), thereby determining the combination of specific groups of microparticles having desired release behavior.

In the groups of microparticles, the composition of the controlled-release polymer coating layer, that is, the kind and the composition ratio of the polymer, may be the same or different, and groups of microparticles in which the average thicknesses of the coating layer are different may be obtained by varying the amount of the coating composition. In the case where the polymer has the same composition, to the same coating layer may be used, thus ensuring process profitability. Particularly useful is the case where the polymer has the same composition.

If the controlled-release polymer coating layer of the groups of microparticles has the same composition, the coating time is adjusted while feeding a predetermined amount of the coating composition, thus sequentially obtaining groups of microparticles having desired average thicknesses of the coating layer. In contrast, if the groups of microparticles have different compositions of the controlled-release polymer coating layer, a first group of microparticles having a desired average thickness of the coating layer is formed with a first coating solution, some of which may then be further coated with a different coating solution from the first coating solution, thus forming another group of microparticles having a different average thickness of the coating layer.

In an embodiment of the invention, the number of groups of microparticles having different average thicknesses of the controlled-release polymer coating layer may be two or more, preferably two or three, and more preferably two.

In the case where two groups of microparticles are provided, a first group of microparticles may have an average thickness of the controlled-release polymer coating layer of 1˜20 μm, and the average thickness ratio of the controlled-release polymer coating layer in the first and second groups of microparticles is 1:1.2˜10, preferably 1:1.8∞7, and more preferably 1:2˜5. In order to enable the microparticles to exhibit different release behaviors, the average thickness of the controlled-release polymer coating layer of the second group of microparticles is preferably 1.2 times the average thickness of the controlled-release polymer coating layer of the first group of microparticles, and is more preferably 10 times or less the average thickness of the controlled-release polymer coating layer of the first group of microparticles so as to maintain the release of the active ingredient at a predetermined concentration to obtain a desired release pattern.

As such, the weight ratio of the first group of microparticles and the second group of microparticles may be adjusted so that the total weight ratio of the active ingredient included in these groups of microparticles falls in the range of 1:0.1˜10, to preferably 1:0.1˜8, more preferably 1:0.1∞6, and much more preferably 1:0.1˜4.

In order to obtain minimum release control effects in the groups of microparticles, the total weight of the active ingredient included in the second group of microparticles is adjusted so as to be at least 0.1 times the total weight of the active ingredient included in the first group of microparticles, and is preferably 10 times or less the total weight of the active ingredient included in the first group of microparticles so as to maintain the release of the active ingredient at a predetermined concentration to obtain a desired release pattern.

On the other hand, in the case where three groups of microparticles are provided, the pharmaceutical composition of the invention may include first and second groups of microparticles, each of the microparticles comprising a core including tamsulosin or pharmaceutically acceptable salts thereof, a controlled-release polymer coating layer formed on the core, and an enteric polymer outer layer formed on the controlled-release polymer coating layer; and a third group of microparticles, each of the microparticles comprising the core mentioned above, a controlled-release polymer coating layer formed on the core, and an enteric polymer outer layer formed on the polymer coating layer, wherein the average thickness of the polymer coating layer of the first and second groups of microparticles is different from that of the third group of microparticles.

In the case where three groups of microparticles are provided, the average thickness of the controlled-release polymer coating layer of the first group of microparticles is 1˜20 μm, the average thickness ratio of the controlled-release polymer coating layer in the first group of microparticles and the second group of microparticles is 1:1.2˜10, preferably 1:1.8˜7, and more preferably 1:2˜5, and the average thickness ratio of the controlled-release polymer coating layer in the second group of microparticles and the third group of microparticles is 1:1.2˜5, preferably 1:1.5˜4, and more preferably 1:1.8˜3.5.

Specifically, in order to allow the groups of microparticles to exhibit different release behaviors, the average thickness of the controlled-release polymer coating layer of the second group of microparticles is at least 1.2 times the average thickness of the controlled-release polymer coating layer of the first group of microparticles, and is preferably 10 times or less the average thickness of the controlled-release polymer coating layer of the first group of microparticles so as to maintain the release of the active ingredient at a predetermined concentration to obtain a desired release pattern. Furthermore, the average thickness ratio of the controlled-release polymer coating layer of the third group of microparticles relative to the second group of microparticles is preferably set in the above range due to the above reasons.

Thus, in the groups of microparticles, the average diameter of microparticles composed exclusively of the core and the controlled-release polymer coating layer formed thereon is 6˜4000 μm, preferably 11˜2500 μm, and more preferably 16˜2000 μm. Particularly in the case of an orally disintegrating tablet or chewable tablet including the pharmaceutical composition of the invention, the average diameter of the microparticles is preferably set to 31˜1500 μm in order to minimize foreign body sensation in the mouth.

As such, the weight ratio of the first group of microparticles, the second group of microparticles, and the third group of microparticles may be adjusted so that the total weight ratio of the active ingredient included in these groups of microparticles falls in the range of 1:0.1˜10:0.1˜10, preferably 1:0.1˜8: 0.1˜8, more preferably 1:0.1˜6:0.1˜6, and much more preferably 1:0.1˜4:0.1˜4. That is, the weight ratio preferably falls in the above range so as to obtain minimum release control effects in the microparticles and to maintain the release of the active ingredient at a predetermined concentration.

To obtain a desired dissolution pattern, the average thickness ratio of the coating layer and the weight ratio of the active ingredient in the groups of microparticles may be variously modified, and a desired release pattern may be ensured in the case of a specific combination.

Enteric Polymer Outer Layer

The enteric polymer outer layer is applied on the controlled-release polymer coating layer mentioned above, so that the release of the active ingredient is controlled to a specific level or less in the gastrointestines at low pH.

According to an embodiment of the invention, the enteric polymer outer layer may be formed on the controlled-release polymer coating layer via a coating process using a typical coater, a fluidized bed coater, a fluidized bed processor, or a fluidized bed granulator. Specifically, this process may be conducted by means of a fluidized bed system with a bottom spray, a centrifugal granulator, Granurex® (available from Freund), etc.

As such, the enteric polymer outer layer may be formed by performing separate coating in each of the groups of microparticles in which the average thicknesses of the controlled-release polymer coating layer are different, or by mixing the groups of microparticles at a specific ratio and then performing coating, and the scope of the invention is not limited by the coating sequence.

The enteric polymer outer layer preferably includes an enteric polymer that is conventionally used in the art.

The enteric polymer means a pH-dependent polymer, that is, a polymer which is stable under acidic conditions of pH of less than 5 and dissolves under weak acidic or neutral conditions of pH 5 or more.

Specifically, the enteric polymer may be one or more selected from the group consisting of an enteric cellulose derivative, an enteric acrylic acid-based copolymer, an enteric maleic acid-based copolymer, and an enteric polyvinyl derivative, and is preferably one or more selected from the group consisting of hydroxypropyl methylcellulose acetate succinate, hydroxypropyl methylcellulose phthalate, hydroxymethyl ethylcellulose phthalate, cellulose acetate phthalate, cellulose acetate succinate, cellulose acetate maleate, cellulose benzoate phthalate, cellulose propionate phthalate, methylcellulose phthalate, carboxymethyl ethylcellulose, ethylhydroxyethyl cellulose phthalate, a styrene.acrylic acid copolymer, a methyl acrylate.acrylic acid copolymer, a methyl acrylate.methacrylic acid copolymer, a butyl acrylate.styrene.acrylic acid copolymer, a methacrylic acid.methyl acrylate copolymer, a methacrylic acid.ethyl acrylate copolymer, a methyl acrylate.methacrylic acid.octyl acrylate copolymer, a vinyl acetate.maleic anhydride copolymer, an ethylene.maleic anhydride copolymer, a vinyl butyl ether.maleic anhydride copolymer, an acrylonitrile.methyl acrylate.maleic anhydride copolymer, a butyl acrylate.styrene.maleic anhydride copolymer, polyvinylalcohol phthalate, polyvinylacetal phthalate, polyvinyl butyrate phthalate, and polyvinylacetoacetal phthalate.

On the other hand, the enteric polymer outer layer may further include one or more selected from the group consisting of a water-insoluble polymer, a water-soluble polymer, and a gastric polymer, in addition to the enteric polymer, depending on the release pattern of the active ingredient by means of the core and the controlled-release polymer coating layer or the form of the preparation.

The additional polymer components are not particularly limited so long as they are conventional in the art.

In an embodiment of the invention, the water-insoluble polymer may be water-insoluble cellulose ether, a water-insoluble acrylic acid-based copolymer, or mixtures thereof, and is preferably one or more selected from the group consisting of ethylcellulose, an ethyl acrylate.methyl methacrylate.trimethylammonioethyl methacrylate chloride copolymer, a methyl methacrylate.ethyl acrylate copolymer, and polyvinylacetate. Particularly, the ethyl acrylate.methylmethacrylate.trimethylammonioethyl methacrylate chloride copolymer preferably may be those having 8.85˜11.96% of ammonio methacrylate units (e.g. Eudragit® RL) or having 4.48˜6.77% of ammonio methacrylate units (e.g. Eudragit® RS), the methylmethacrylate.ethyl acrylate copolymer may be Eudragit® NE30D, Eudragit® NE40D, Eudragit® NM30D, or mixtures thereof, and the polyvinylacetate may be Kollicoat® SR30D.

Also, the water-soluble polymer may be one or more selected from the group consisting of water-soluble cellulose ether, a water-soluble polyvinyl derivative, and an alkylene oxide polymer, and is preferably one or more one or more selected from the group consisting of methylcellulose, hydroxypropyl cellulose, hydroxypropyl methylcellulose, polyvinylpyrrolidone, polyvinyl alcohol, polyethylene glycol, and polypropylene glycol.

Also, the gastric polymer may be one or more selected from the group consisting of a gastric polyvinyl derivative and a gastric acrylic acid-based copolymer, and is preferably one or more selected from the group consisting of polyvinylacetal diethylamino acetate, and a methyl methacrylate.butyl methacrylate.dimethylaminoethyl methacrylate copolymer.

On the other hand, in order to achieve additional purposes including coating efficiency, stability of active ingredient, outer appearance, color, protection, maintenance, coupling, improvements in performance and manufacturing process, or supplemental release control upon forming the enteric polymer coating layer, a variety of biologically inert ingredients may be additionally used.

The biologically inert ingredient may include a saccharide, a sugar alcohol, a polymer, a colorant, a flavorant, a sweetener, a surfactant, a lubricant, a stabilizer, an antioxidant, a foaming agent, paraffin, wax, or a plasticizer, and the detailed description thereof refers to the above description of the core.

The enteric polymer outer layer has an average thickness of 2˜500 μm, preferably 3˜400 μm, and more preferably 4∞250 μm. In order to exhibit the release control effect of the active ingredient by the enteric polymer outer layer in the gastrointestines at low pH, the average thickness of the enteric polymer outer layer is preferably set to 2 μm or more, and is preferably set to 500 μm or less to effectively release the active ingredient in the intestines.

Accordingly, the microparticles of the pharmaceutical composition of the invention (i.e. microparticles each comprising a core including an active ingredient, and a controlled-release polymer coating layer and an enteric polymer outer layer sequentially formed on the core) have a final average diameter of 10˜4500 μm, preferably 15˜3000 μm, and more preferably 20˜2500 μm. Particularly in the case of an orally disintegrating tablet or chewable tablet including the pharmaceutical composition of the invention, the final average diameter of the microparticles is set to 35˜2000 μm, and preferably 35˜800 μm, in order to minimize foreign body sensation in the mouth.

Dissolution Pattern of Pharmaceutical Composition

The groups of microparticles included in the pharmaceutical composition of the invention are configured as above, and may thus exhibit the following dissolution pattern.

In the case where two groups of microparticles in which the average thicknesses of the controlled-release polymer coating layer are different are provided, each of the groups of microparticles shows the following dissolution pattern when being tested according to a dissolution test method of Korean Pharmacopoeia 8^{th} revision (KP VIII) (a second paddle method: 100 rotations per min, 500 ml of dissolution solutions at pH 1.2 and pH 7.2).

a) The first group of microparticles:

With respect to a dissolution solution at pH 1.2, the contained active ingredient is dissolved in an amount of 30 wt % (preferably 20 wt %) or less within 2 h, based on the total weight thereof.

With respect to a dissolution solution at pH 7.2, the contained active ingredient is dissolved in an amount of 70 wt % (preferably 60 wt %) or less within 30 min, and 95 wt % (preferably 90 wt %) or more within 4 h, based on the total weight thereof.

b) The second group of microparticles:

With respect to a dissolution solution at pH 1.2, the contained active ingredient is dissolved in an amount of 20 wt % (preferably 10 wt %) or less within 2 h, based on the total weight thereof.

With respect to a dissolution solution at pH 7.2, the contained active ingredient is dissolved in an amount of 50 wt % (preferably 30 wt %) or less within 30 min, and 90 wt % (preferably 80 wt %) or more within 4 h, based on the total weight thereof.

In addition, in the case where two groups of microparticles in which the average thicknesses of the controlled-release polymer coating layer are different are provided, the case where these two groups of microparticles are mixed so that the total to weight ratio of the active ingredient included in the groups of microparticles falls in the range of 1:0.1˜10, preferably 1:0.1˜8, more preferably 1:0.1˜6, and much more preferably 1:0.1˜4 shows the following dissolution pattern when being tested according to a dissolution test method of Korean Pharmacopoeia 8^{th} revision (KP VIII) (a second paddle method: 100 rotations per min, 500 ml of dissolution solutions at pH 1.2 and pH 7.2).

a) With respect to a dissolution solution at pH 1.2:

The active ingredient is dissolved in an amount of 1˜30 wt % (preferably 5˜15 wt %) within 2 h, based on the total weight thereof

b) With respect to a dissolution solution at pH 7.2:

The active ingredient is dissolved in an amount of 10˜60 wt % (preferably 17˜55 wt %) within 30 min, based on the total weight thereof.

The active ingredient is dissolved in an amount of 30˜80 wt % (preferably 30˜75 wt %) within 1 h, based on the total weight thereof.

The active ingredient is dissolved in an amount of 50 wt % (preferably 80 wt %) or more within 4 h, based on the total weight thereof.

In the case where three groups of microparticles in which the average thicknesses of the controlled-release polymer coating layer are different are provided, the third group of microparticles shows the following dissolution pattern when being tested under the same conditions as above.

With respect to a dissolution solution at pH 1.2, the active ingredient is dissolved in an amount of 10 wt % (preferably 5 wt %) or less within 2 h, based on the total weight thereof.

With respect to a dissolution solution at pH 7.2, the active ingredient is dissolved in an amount of 30 wt % (preferably 10 wt %) or less within 30 min, and 70 wt % (preferably 60 wt %) or more within 4 h, based on the total weight thereof.

Also, in the case where three groups of microparticles in which the average thicknesses of the controlled-release polymer coating layer are different are provided, the case where these three groups of microparticles are mixed so that the total weight ratio of the active ingredient included in the groups of microparticles falls in the range of 1:0.1˜10:0.1˜10, preferably 1:0.1˜8:0.1˜8, more preferably 1:0.1˜6:0.1˜6, and much more preferably 1:0.1˜4:0.1˜4 shows the same dissolution pattern as in the above two groups of microparticles, when being tested according to a dissolution test method of Korean Pharmacopoeia 8^{th} revision (KP VIII) (a second paddle method: 100 rotations per min, 500 ml of dissolution solutions at pH 1.2 and pH 7.2).

Therefore, the composition of the invention enables the active ingredient to be controlled-released for a long period of time so as to sustain the efficacy thereof.

II. Oral formulation Including the Pharmaceutical Composition

According to another embodiment of the present invention, an oral formulation including the controlled-release pharmaceutical composition as above is provided.

The oral formulation of the invention may include tamsulosin at 0.2˜0.8 mg per unit dosage form, and may exhibit an optimal active ingredient dissolution pattern even when taken once a day.

Upon oral administration, the oral formulation of the invention may effectively control the drug release in the stomach, and may inhibit drastic drug release upon initial administration (preferably 30% or less of the administered drug is released for 2 h), thereby reducing side-effects such as orthostatic dysfunction or the like due to a drastic increase in initial blood concentration of the active ingredient. Also, even after the oral formulation passes through the gastrointestines at low pH and is transferred to the small intestine at approximately neutral pH, it may exhibit controlled-release behavior for a predetermined period of time (preferably 6 h or longer), and the drug may be sufficiently released in the small intestine which is the drug absorption portion, thus maximizing the efficacy.

The oral formulation may be formulated into conventional forms without particular limitation in the art.

According to an embodiment of the invention, the oral formulation may be a capsule, a tablet (a normal tablet, a double layer tablet, a chewable tablet, or an orally disintegrating tablet), a dry syrup formulation, a syrup, a jelly formulation, or a granule, and is preferably a capsule or a tablet, and is more preferably an orally disintegrating tablet.

In the course of manufacturing the oral formulation, not only the controlled-release pharmaceutical composition of the invention, but also an additive, such as an excipient, a disintegrant, a binder, a lubricant, a colorant, a flavorant, a sweetener, a surfactant, a stabilizer, a foaming agent, an antioxidant, etc., may be added, depending on the dosage form of the preparation.

In the embodiment of the invention, the capsule may be manufactured by mixing the controlled-release pharmaceutical composition of the invention with a lubricant, an excipient, etc., and filling a hard capsule with the mixture; the normal tablet or chewable tablet may be manufactured by tabletting the controlled-release pharmaceutical composition of the invention together with an excipient, a disintegrant, a binder, a lubricant, a colorant, a flavorant, a sweetener, etc.; and the syrup may be manufactured by uniformly dispersing the controlled-release pharmaceutical composition of the invention in a syrup and preventing the active ingredient from being released into the syrup during storage.

In the case of an orally disintegrating tablet which is improved in convenience of drug taking, it may disintegrate in the mouth and may be taken without water, and thus may be easily applied to elderly patients, patients suffering from dysphagia, patients who have difficulty swallowing a tablet, etc. Also, in the case of an orally disintegrating tablet, the dissolution pattern of the active ingredient is controlled by the microparticles included in the pharmaceutical composition of the invention, thus exhibiting optimal efficacy.

In particular, because an orally disintegrating tablet stays in the mouth for a while when taken, the bitter taste of tamsulosin has to be shielded. As such, the bitter taste may not be sufficiently shielded only by the addition of a sweetener, a flavorant, etc. However, the case where the controlled-release pharmaceutical composition of the invention is used is advantageous because the release of tamsulosin is suppressed for 1 min or longer (preferably 3 min or longer) in the mouth, thus enabling taking of such a drug without the feel of rejection. The orally disintegrating tablet may be prepared by mixing a known orally disintegrating component, for example, WOWTAB®, Zydis®, OraSolv®, DuraSolv®, QuickSolv®, FlashTab®, AdvaTab®, Lyoc®, FlashDose®, Frosta®, or the like with the controlled-release pharmaceutical composition of the invention.

In the course of forming such a preparation, in order to prevent damage to the controlled-release polymer coating layer of the microparticles included in the pharmaceutical composition of the invention, it is preferred that an appropriate level of tabletting pressure be applied or a buffering material be used.

Also, in the course of forming such a preparation, the controlled-release pharmaceutical composition of the invention may be directly tabletted by being mixed with an excipient without granulation; the above pharmaceutical composition may be first granulated, mixed with an additional excipient, and then tabletted; or the above pharmaceutical composition may be mixed with an excipient, granulated, and then tabletted.

### Advantageous Effect of the Invention

According to the present invention, a controlled-release pharmaceutical composition can easily control the extent of release of an active ingredient depending on changes in pH in the intestinal tract and the release pattern of the active ingredient in the small intestine, thus preventing the active ingredient from being rapidly transferred into the blood to thereby minimize side-effects, and maintaining the effective blood concentration of the active ingredient for a predetermined period of time. Furthermore, this composition can shield the bitter taste of the active ingredient even when exposed to the inside of the mouth, thus increasing the therapeutic effects for patients upon oral administration.

### Mode for Practicing the Invention

A better understanding of the present invention may be obtained through the following examples which are set forth to illustrate, but are not to be construed as limiting the present invention.

The analytical methods performed in the following examples are described below.

Dissolution Test

The dissolution test of the active ingredient of microparticles, tablets, capsules, chewable tablets, and orally disintegrating tablets was conducted according to the second paddle method of “36. Dissolution test method” of Korean Pharmacopoeia 8^{th} revision (KP VIII). As the dissolution solution, a buffer solution at pH 1.2 and a buffer solution at pH 7.2 were used in an amount of 500 ml, and 100 rotations per min were applied.

After dissolution, analysis was conducted using high performance liquid chromatography (HPLC). Specifically, to analyze tamsulosin, 8.7 ml of perchloric acid and 3.0 g of sodium hydroxide were dissolved in 1900 ml of water, the pH of the solution was adjusted to 2.0 using a sodium hydroxide solution, and the amount thereof was adjusted to 2000 ml using water. 1400 ml of this solution was mixed with 600 ml of acetonitrile, and the resulting mixture was used as a mobile phase. Also, a column having an inner diameter of 4.6 mm and a length of 150 mm and filled with 5 μm of octadecyl silylated silica gel was used, the flow rate of the mobile phase was 1.0 ml/min, the amount of the sample was 100 μl, the detection wavelength was 225 nm, and the measurement time was 12 min.

Content Test

To assay the content of the active ingredient included in microparticles, tablets, capsules, chewable tablets, and orally disintegrating tablets, the mobile phase (obtained by dissolving 8.7 ml of perchloric acid and 3.0 g of sodium hydroxide in 1900 ml of water, adjusting the pH of the resulting solution to 2.0 using a sodium hydroxide solution, adjusting the amount of the solution to 2000 ml, and mixing 1400 ml of the solution with 600 ml of acetonitrile) used in the above dissolution test was added with microparticles containing the active ingredient, mixed while shaking, and centrifuged to obtain a supernatant, and this solution was filtered and diluted to make a test solution which was then analyzed using HPLC. A column having an inner to diameter of 4.6 mm and a length of 150 mm and filled with 5 μm of octadecyl silylated silica gel was used, the flow rate of the mobile phase was 1.0 ml/min, the amount of the sample was 100 μl, the detection wavelength was 225 nm, and the measurement time was 12 min.

Measurement of Hardness

In the case of a preparation such as a chewable tablet and an orally disintegrating tablet, the hardness of the preparation was measured using a hardness tester 8M (Dr. Schleuniger, Switzerland), and the hardness values of at least six test samples were measured and averaged.

Disintegration Test

In the case of an orally disintegrating tablet, the disintegration test of the orally disintegrating tablet in the mouths of volunteers was conducted. The volunteers were randomly chosen and their mouths were washed with water. As soon as the tablet was placed on the tongue of the volunteers, the disintegration time was measured using a stopwatch. The volunteers were allowed to move the orally disintegrating tablet to the ceiling of the mouth using their tongue and to gently roll the tablet without biting it or to roll it from side to side. When the tablet was collapsed and could be swallowed with saliva, the stopwatch was immediately stopped and the time was recorded.

### EXAMPLE

### Example 1

### Formation of Core Containing Tamsulosin

Cores in the form of a pharmaceutically acceptable inert seed being coated with an active ingredient were manufactured as follows.

Specifically, about 14.40 g of tamsulosin hydrochloride, about 14.40 g of hydroxypropyl methylcellulose, and about 4.32 g of talc were dissolved in about 2016 g of a solvent (comprising water and ethanol mixed at 5:2), thus preparing a coating solution.

About 1800.0 g of microcrystalline cellulose CP102 (particle size distribution 106˜212 μm, Celphere®, Asahi Kasei, Japan) as an inert seed was placed in a fluidized bed coater GPCG-1 (Glatt, Germany), and the prepared coating solution was sprayed in a bottom spray mode to perform coating. After completion of the spraying of the coating solution, drying was conducted, thus obtaining about 1833.0 g of cores containing tamsulosin.

The cores were measured to contain about 0.79% of tamsulosin using the above amount test method (HPLC), and mostly passed through a 250 μm sieve, and the average diameter of the cores was observed to be 150˜220 μm using SEM.

### Example 2-A

### Formation of Controlled-Release Coating Layer A on the Core

About 60 g of Eudragit® RS 100 (Evonik) was dissolved in a solvent mixture comprising about 450 g of ethanol and about 150 g of water, and about 3 g of triethyl citrate and about 18 g of talc were added, thus preparing a controlled-release coating solution A.

About 611 g of the cores of Example 1 were placed in a fluidized bed coater, and the controlled-release coating solution A was sprayed in a bottom spray mode to perform coating. After completion of the spraying of the coating solution, drying was conducted, thus obtaining about 692 g of a group of microparticles A each comprising the controlled-release coating layer formed on the core.

The group of microparticles A was measured to contain about 0.69% of tamsulosin using the above amount test method (HPLC), and mostly passed through a 300 μm sieve, and the average thickness of the controlled-release polymer coating layer A was observed to be about 3.4 μm using SEM.

### Example 2-B

### Formation of Controlled-release Coating Layer B on the Core

About 180 g of Eudragit® RS I00 (Evonik) was dissolved in a solvent mixture comprising about 1350 g of ethanol and about 450 g of water, and about 9 g of triethyl citrate and about 54 g of talc were added, thus preparing a controlled-release coating solution B.

About 611 g of the cores of Example I were placed in a fluidized bed coater, and the controlled-release coating solution B was sprayed in a bottom spray mode to perform coating. After completion of the spraying of the coating solution, drying was conducted, thus obtaining about 854 g of a group of microparticles B each comprising the controlled-release coating layer formed on the core.

The group of microparticles B was measured to contain about 0.56% of tamsulosin using the above amount test method (HPLC), and mostly passed through a 300 μm sieve, and the average thickness of the controlled-release polymer coating layer B was observed to be about 11.0 μm using SEM.

### Example 2-C

### Formation of Controlled-Release Coating Layer C on the Core

About 300 g of Eudragit® RS 100 (Evonik) was dissolved in a solvent mixture comprising about 2250 g of ethanol and about 750 g of water, and about 15 g of triethyl citrate and about 90 g of talc were added, thus preparing a controlled-release coating solution C.

About 611 g of the cores of Example I were placed in a fluidized bed coater, and the controlled-release coating solution C was sprayed in a bottom spray mode to perform coating. After completion of the spraying of the coating solution, drying was conducted, thus obtaining about 1016 g of a group of microparticles C each comprising the controlled-release coating layer formed on the core.

The group of microparticles C was measured to contain about 0.47% of tamsulosin using the above amount test method (HPLC), and mostly passed through a 300 μm sieve, and the average thickness of the controlled-release polymer coating layer C was observed to be about 17.2 μm using SEM.

### Example 3-A

### Formation of Enteric Polymer Outer Layer A on the Controlled-Release Coating Layer A

About 200 g of Eudragit® L 30D-55 (Evonik) and about 200 g of Eudragit® NE 30D (Evonik) were respectively diluted with about 200 g of water and mixed, and a solution of about 7.96 g of triethyl citrate, about 1.97 g of polysorbate80, and about 4.64 g of glycerin monostearate dispersed in about 62 g of water was added thereto, thus preparing an enteric polymer outer layer coating solution A.

About 692 g of the group of microparticles A of Example 2-A was placed in a fluidized bed coater, and the enteric polymer outer layer coating solution A was sprayed in a bottom spray mode to perform coating. After completion of the spraying of the coating solution, drying was conducted, thus obtaining about 826 g of a group of microparticles A each comprising the enteric polymer outer layer formed on the controlled-release coating layer.

The group of microparticles A was measured to contain about 0.58% of tamsulosin using the above amount test method (HPLC), and mostly passed through a 300 μm sieve, and the average thickness of the enteric polymer outer layer A was observed to be about 6.6 μm using SEM.

### Example 3-B

### Formation of Enteric Polymer Outer Layer B on the Controlled-Release Coating Layer B

About 200 g of Eudragit® L 30D-55 (Evonik) and about 200 g of Eudragit® NE 30D (Evonik) were respectively diluted with about 200 g of water and mixed, and a solution of about 7.96 g of triethyl citrate, about 1.97 g of polysorbate80, and about 4.64 g of glycerin monostearate dispersed in about 62 g of water was added thereto, thus preparing an enteric polymer outer layer coating solution B.

About 854 g of the group of microparticles B of Example 2-B was placed in a fluidized bed coater, and the enteric polymer outer layer coating solution B was sprayed in a bottom spray mode to perform coating. After completion of the spraying of the coating solution, drying was conducted, thus obtaining about 988 g of a group of microparticles B each comprising the enteric polymer outer layer formed on the controlled-release coating layer.

The group of microparticles B was measured to contain about 0.49% of tamsulosin using the above amount test method (HPLC), and mostly passed through a 300 μm sieve, and the average thickness of the enteric polymer outer layer B was observed to be about 5.9 μm using SEM.

### Example 3-C

### Formation of Enteric Polymer Outer Layer C on the Controlled-Release Coating Layer C

About 200 g of Eudragit® L 30D-55 (Evonik) and about 200 g of Eudragit® NE 30D (Evonik) were respectively diluted with about 200 g of water and mixed, and a solution of about 7.96 g of triethyl citrate, about 1.97 g of polysorbate80, and about 4.64 g of glycerin monostearate dispersed in about 62 g of water was added thereto, thus preparing an enteric polymer outer layer coating solution C.

About 1016 g of the group of microparticles C of Example 2-C was placed in a fluidized bed coater, and the enteric polymer outer layer coating solution C was sprayed in a bottom spray mode to perform coating. After completion of the spraying of the coating solution, drying was conducted, thus obtaining about 1150 g of a group of microparticles C each comprising the enteric polymer outer layer formed on the controlled-release coating layer.

The group of microparticles C was measured to contain about 0.42% of tamsulosin using the above amount test method (HPLC), and mostly passed through a 300 μm sieve, and the average thickness of the enteric polymer outer layer C was observed to be about 5.4 μm using SEM.

### Example 4

### Measurement of Dissolution Pattern of Each of Groups of Microparticles A, B, and C

According to the above dissolution test method, the dissolution pattern of each of the groups of microparticles A and B of Examples 3-A and 3-B was measured. About 34.70 mg each of the group of microparticles A of Example 3-A was precisely weighed to make six samples, 500 ml of each of buffer solutions at pH 1.2 and pH 7.2 was placed in a six-stage dissolution tester, and a dissolution test was conducted according to a paddle method (100 rotations per min) for 2 h (pH 1.2) and 6 h (pH 7.2). The HPLC analytical results were averaged, thus obtaining dissolution data. The results are shown in Tables 1 and 2 below.

In addition, about 41.50 mg each of the group of microparticles B of Example 3-B was precisely weighed and the dissolution test thereof was performed under the same conditions as in the group of microparticles A. The results are shown in Tables 1 and 2 below.

TABLE 1

| | Dissolution (%) of active ingredient | | |
|---|---|---|---|
| | in buffer solution at pH 1.2 | | |
| | Group of micro- | Group of micro- | Group of micro- |
| Time | particles A | particles B | particles C |
| 2 h | 15.4 | 7.0 | 2.1 |

TABLE 2

| | Dissolution (%) of active ingredient | | |
|---|---|---|---|
| | in buffer solution at pH 7.2 | | |
| | Group of micro- | Group of micro- | Group of micro- |
| Time | particles A | particles B | particles C |
| 30 min | 57.0 | 6.7 | 0.9 |
| 1 h | 78.5 | 18.8 | 6.6 |
| 2 h | 98.0 | 57.0 | 32.9 |
| 4 h | 98.5 | 89.8 | 72.3 |
| 6 h | 99.0 | 95.7 | 80.0 |

### Example 5

### Measurement of Dissolution Pattern of Combination of Groups of Microparticles A and B

According to the dissolution test method, the dissolution pattern of the combination of the groups of microparticles A and B of Examples 3-A and 3-B was measured.

The weight ratio of the group of microparticles A of Example 3-A and the group of microparticles B of Example 3-B was precisely set so that the total weight ratio of the active ingredient included in these two groups of microparticles was 1:0.1, 1:1, and 1:4, thus making six combination samples, and 500 ml of each of buffer solutions at pH 1.2 and pH 7.2 was placed in a six-stage dissolution tester. A dissolution test was then conducted according to a paddle method (100 rotations per min) for 2 h and 6 h. The HPLC analytical results were averaged, thus obtaining dissolution data, and the results are shown in Table 3 below.

TABLE 3

| | | Dissolution (%) of active ingredient by | | |
|---|---|---|---|---|
| pH of buffer | | combination of groups of microparticles | | |
| solution | Time | A:B = 1:0.1 | A:B = 1:1 | A:B = 1:4 |
| 1.2 | 2 h | 14.7 | 10.1 | 8.9 |
| 7.2 | 30 min | 53.4 | 32.3 | 17.0 |
| | 1 h | 74.2 | 48.0 | 31.2 |
| | 2 h | 94.5 | 79.3 | 66.3 |
| | 4 h | 97.7 | 94.9 | 92.1 |
| | 6 h | 99.2 | 97.8 | 96.5 |

As is apparent from Table 3, in the case of the combination of the groups of microparticles A and B of Examples 3-A and 3-B, excessive release could be to prevented, as well as initial efficacy manifestation, in the buffer solution at pH 1.2. In particular, the active ingredient was gradually released for 6 h in the buffer solution at pH 7.2, so that the efficacy thereof could be sustained.

### Example 6

### Measurement of Dissolution Pattern of Combination of Groups of Microparticles A, B, and C

According to the dissolution test method, the dissolution pattern of the combination of the groups of microparticles A, B, and C of Examples 3-A, 3-B, and 3-C was measured.

The weight ratio of the group of microparticles A of Example 3-A, the group of microparticles B of Example 3-B, and the group of microparticles C of Example 3-C was precisely set so that the total weight ratio of the active ingredient included in these three groups of microparticles was 1:0.1:0.1, 1:1:1.6, and 1:4:0.1, thus making six combination samples, and 500 ml of each of buffer solutions at pH 1.2 and pH 7.2 was placed in a six-stage dissolution tester. A dissolution test was conducted according to a paddle method (100 rotations per min) for 2 h and 6 h. The HPLC analytical results were averaged, thus obtaining dissolution data, and the results are shown in Table 4 below.

TABLE 4

| | | Dissolution (%) of active ingredient by | | |
|---|---|---|---|---|
| | | combination of groups of microparticles | | |
| pH of buffer | | A:B:C = | A:B:C = | A:B:C = |
| solution | Time | 1:0.1:0.1 | 1:1:1.6 | 1:4:0.1 |
| 1.2 | 2 h | 2.1 | 13.6 | 7.3 |
| 7.2 | 30 min | 48.5 | 18.2 | 16.9 |
| | 1 h | 67.2 | 31.3 | 32.5 |
| | 2 h | 86.3 | 58.4 | 65.1 |
| | 4 h | 94.3 | 84.9 | 91.4 |
| | 6 h | 98.5 | 91.5 | 96.0 |

As is apparent from Table 4, in the case of the combination of the groups of microparticles A, B, and C of Examples 3-A, 3-B, and 3-C, excessive release could be prevented, as well as initial efficacy manifestation, in the buffer solution at pH 1.2. In particular, the active ingredient was gradually released for 6 h in the buffer solution at pH 7.2, whereby the efficacy thereof could be sustained.

### Example 7

### Formulation of Orally Disintegrating tablet

130 g of Mannogem EZ (spray dried mannitol, SP1) and about 10 g of Advantose FS 95 (spray dried fructose, SPI) were mixed, and granulated with about Is 32.6 g of a 50% sucrose solution (ethanol:water=4:6). The granulated particles were passed through a 600 μm sieve, and the resulting granules were dried in an oven at 50° C.

About 153.4 g of the granules thus prepared, about 17.35 g of the group of microparticles A of Example 3-A, and about 20.75 g of the group of microparticles B of Example 3-B were mixed together, and about 6 g of a disintegrant (Explotab) and about 2.5 g of a lubricant were additionally mixed, followed by tabletting the resulting mixture, thus obtaining about 200 mg of an orally disintegrating tablet.

According to the hardness measurement method and the disintegration test method, the orally disintegrating tablet thus obtained was measured to have a hardness of about 4.5 Kp, and the disintegration time in the mouth was about 13 sec.

1. A pharmaceutical composition comprising a first group of microparticles and a second group of microparticles, each of the microparticles comprising:
a core comprising tamsulosin or pharmaceutically acceptable salts thereof;
a controlled-release polymer coating layer formed on the core; and
an enteric polymer outer layer formed on the controlled-release polymer coating layer,
wherein an average thickness of the controlled-release polymer coating layer is different between the first group of microparticles and the second group of microparticles.

2. The pharmaceutical composition of claim 1, wherein the first group of microparticles has an average thickness of the controlled-release polymer coating layer of 1˜20 μm, and an average thickness ratio of the controlled-release polymer coating layer in the first group of microparticles and the second group of microparticles is 1: 1.2˜10.

3. The pharmaceutical composition of claim 1, wherein a weight ratio of the first group of microparticles and the second group of microparticles is adjusted so that a total weight ratio of tamsulosin or pharmaceutically acceptable salts thereof comprised in the first group of microparticles and the second group of microparticles falls in a range of 1:0.1˜10.

4. The pharmaceutical composition of claim 1, further comprising a third group of microparticles, each of the microparticles comprising:
a core including tamsulosin or pharmaceutically acceptable salts thereof;
a controlled-release polymer coating layer formed on the core; and
an enteric polymer outer layer formed on the controlled-release polymer coating layer,
wherein an average thickness of the controlled-release polymer coating layer of the third group of microparticles is different from the average thickness of the controlled-release polymer coating layer of the first group of microparticles and the second group of microparticles.

5. The pharmaceutical composition of claim 4, wherein the first group of microparticles has the average thickness of the controlled-release polymer coating layer of 1˜20 μm,
an average thickness ratio of the controlled-release polymer coating layer in the first group of microparticles and the second group of microparticles is 1:1.2˜10, and an average thickness ratio of the controlled-release polymer coating layer in the second group of microparticles and the third group of microparticles is 1:1.2˜5.

6. The pharmaceutical composition of claim 4, wherein a weight ratio of the first group of microparticles, the second group of microparticles, and the third group of microparticles is adjusted so that a total weight ratio of tamsulosin or pharmaceutically acceptable salts thereof included in the first group of microparticles, the second group of microparticles, and the third group of microparticles falls in a range of 1:0.1˜10:0.1˜10.

7. The pharmaceutical composition of claim 1, wherein an average diameter of each of the groups of microparticles is 10˜4500 μm.

8. The pharmaceutical composition of claim 1, wherein the core is formed by mixing tamsulosin or pharmaceutically acceptable salts thereof with an inert seed, by incorporating tamsulosin or pharmaceutically acceptable salts thereof in the inert seed, or by coating a surface of the inert seed with tamsulosin or pharmaceutically acceptable salts thereof.

9. The pharmaceutical composition of claim 1, wherein the controlled-release polymer coating layer comprises a water-insoluble polymer.

10. The pharmaceutical composition of claim 9, wherein the water-insoluble polymer is one or more selected from the group consisting of ethylcellulose, an ethyl acrylate/methyl methacrylate/trimethylammonioethyl methacrylate chloride copolymer, a methyl methacrylate/ethyl acrylate copolymer, and polyvinylacetate.

11. The pharmaceutical composition of claim 9, wherein the controlled-release polymer coating layer further comprises one or more selected from the group consisting of:
water-soluble polymers comprising methyl cellulose, hydroxypropyl cellulose, hydroxypropyl methylcellulose, polyvinylpyrrolidone, polyvinyl alcohol, polyethylene glycol, and polypropylene glycol;
enteric polymers comprising hydroxypropyl methylcellulose acetate succinate, hydroxypropyl methylcellulose phthalate, hydroxymethyl ethylcellulose phthalate, cellulose acetate phthalate, cellulose acetate succinate, cellulose acetate maleate, cellulose benzoate phthalate, cellulose propionate phthalate, methylcellulose phthalate, carboxylmethyl ethylcellulose, ethylhydroxyethyl cellulose phthalate, a styrene/acrylic acid copolymer, a methyl acrylate/acrylic acid copolymer, a methyl acrylate/methacrylic acid copolymer, a butyl acrylate/styrene.acrylic acid copolymer, a methacrylic acid/methyl acrylate copolymer, a methacrylic acid/ethyl acrylate copolymer, a methyl acrylate.methacrylic acid/octyl acrylate copolymer, a vinyl acetate/maleic anhydride copolymer, an ethylene/maleic anhydride copolymer, a vinyl butyl ether/maleic anhydride copolymer, an acrylonitrile/methyl acrylate.maleic anhydride copolymer, a butyl acrylate/styrene.maleic anhydride copolymer, polyvinyl alcohol phthalate, polyvinylacetal phthalate, polyvinylbutyrate phthalate, and polyvinylacetoacetal phthalate; and
gastric polymers comprising polyvinylacetal diethylamino acetate, and a methyl methacrylate/butyl methacrylate/dimethylaminoethyl methacrylate copolymer.

12. The pharmaceutical composition of claim 1, wherein the controlled-release polymer coating layer of each of the groups of microparticles has a same polymer composition.

13. The pharmaceutical composition of claim 1, wherein the enteric polymer outer layer comprises one or more selected from the group consisting of hydroxypropyl methylcellulose acetate succinate, hydroxypropyl methylcellulose phthalate, hydroxymethyl ethylcellulose phthalate, cellulose acetate phthalate, cellulose acetate succinate, cellulose acetate maleate, cellulose benzoate phthalate, cellulose propionate phthalate, methylcellulose phthalate, carboxylmethyl ethylcellulose, ethylhydroxyethyl cellulose phthalate, a styrene/acrylic acid copolymer, a methyl acrylate/acrylic acid copolymer, a methyl acrylate/methacrylic acid copolymer, a butyl acrylate.styrene/acrylic acid copolymer, a methacrylic acid/methyl acrylate copolymer, a methacrylic acid/ethyl acrylate copolymer, a methyl acrylate/methacrylic acid/octyl acrylate copolymer, a vinyl acetate/maleic anhydride copolymer, an ethylene/maleic anhydride copolymer, a vinyl butyl ether/maleic anhydride copolymer, an acrylonitrile/methyl acrylate/maleic anhydride copolymer, a butyl acrylate/styrene.maleic anhydride copolymer, polyvinyl alcohol phthalate, polyvinylacetal phthalate, polyvinylbutyrate phthalate, and polyvinylacetoacetal phthalate.

14. The pharmaceutical composition of claim 13, wherein the enteric polymer outer layer further comprises one or more selected from the group consisting of:
water-insoluble polymers comprising ethylcellulose, an ethyl acrylate/methyl methacrylate/trimethylammonioethyl methacrylate chloride copolymer, a methyl methacrylate/ethyl acrylate copolymer, and polyvinylacetate;
water-soluble polymers comprising methylcellulose, hydroxypropyl cellulose, hydroxypropyl methylcellulose, polyvinylpyrrolidone, polyvinyl alcohol, polyethylene glycol, and polypropylene glycol; and
gastric polymers comprising polyvinylacetal diethylamino acetate and a methyl methacrylate/butyl methacrylate/dimethylaminoethyl methacrylate copolymer.

15. The pharmaceutical composition of claim 1, wherein one or more of the core, the controlled-release polymer coating layer, and the enteric polymer outer layer further comprise one or more selected from the group consisting of a saccharide, a sugar alcohol, a water-soluble polymer, a water-insoluble polymer, an enteric polymer, a gastric polymer, a colorant, a fiavorant, a sweetener, a surfactant, a lubricant, a stabilizer, an antioxidant, a foaming agent, paraffin, wax, an antifoaming agent, and a plasticizer.

16. The pharmaceutical composition of claim 15, wherein the plasticizer is one or more selected from the group consisting of triethyl citrate, dibutyl phthalate, diethyl phthalate, dibutyl sebacate, diethyl sebacate, tributyl citrate, acetyl triethyl citrate, acetyl triethyl citrate, propylene glycol, triacetin, polyethylene glycol, cetyl alcohol, stearyl alcohol, and cetostearyl alcohol.

17. The pharmaceutical composition of claim 1, which has a dissolution pattern in which, according to a dissolution test method of Korean Pharmacopoeia 8^{th} revision (KP VIII) (a second paddle method: 100 rotations per min, 500 ml of dissolution solutions at pH 1.2 and pH 7.2):
a) with respect to a dissolution solution at pH 1.2, the tamsulosin or pharmaceutically acceptable salts thereof are dissolved in an amount of 1˜30 wt % within 2 h, based on a total weight thereof; and
b) with respect to a dissolution solution at pH 7.2, the tamsulosin or pharmaceutically acceptable salts thereof are dissolved in an amount of 10˜60 wt % within 30 min, 30˜80 wt % within 1 h, and 50 wt % or more within 4 h, based on the total weight thereof.

18. The pharmaceutical composition of claim 1, which has a dissolution pattern in which, according to a dissolution test method of Korean Pharmacopoeia 8^{th} revision (KP VIII) (a second paddle method: 100 rotations per min, 500 ml of dissolution solutions at pH 1.2 and pH 7.2):
a) with respect to a dissolution solution at pH 1.2, the tamsulosin or pharmaceutically acceptable salts thereof are dissolved in an amount of 5˜15 wt % within 2 h, based on a total weight thereof; and
b) with respect to a dissolution solution at pH 7.2, the tamsulosin or pharmaceutically acceptable salts thereof are dissolved in an amount of 17˜55 wt % within 30 min, 30˜75 wt % within 1 h, and 80 wt % or more within 4 h, based on the total weight thereof.

19. An oral formulation comprising the pharmaceutical composition of claim 1.

20. The oral formulation of claim 19, which is a capsule, a normal tablet, a double layer tablet, a chewable tablet, an orally disintegrating tablet, a dry syrup formulation, a syrup, a jelly formulation, or a granule.
